(19) United States
(12) Patent Application Publication
(10) Pub. No.: US 2008/0194446 A1
(43) Pub. Date: Aug. 14, 2008

(54) Use of Dibenzanthrone and Isodibenzanthrone Derivatives as Marking Substances for Liquids

(75) Inventors: Sophia Ebert, Mannheim (DE); Rudiger Sens, Ludwigshafen (DE); Christos Vamvakaris, Mannheim (DE); Wolfgang Ahlers, Worms (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 11/817,325

(22) PCT Filed: Mar. 9, 2006
(86) PCT No.: PCT/EP06/60609

§ 371 Date: Aug. 29, 2007

(30) Foreign Application Priority Data
Mar. 15, 2005 (DE) 10 2005 012 211.6

(51) Int. Cl. C10M 129/24; C10L 1/18; C07C 49/115

(52) U.S. Cl. 508/578; 568/326

(57) ABSTRACT

The present invention relates to the use of selected dibenzanthrone or isodibenzanthrone derivatives as markers for liquids, in particular for mineral oils. The present invention further relates both to fuel and lubricant additive concentrates and to mineral oils which comprise at least one such dibenzanthrone or isodibenzanthrone derivative.

The present invention relates to the use of dibenzanthrone derivatives of the general formula Ia

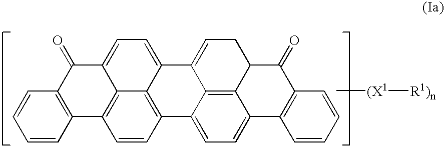

or isodibenzanthrone derivatives of the general formula Ib

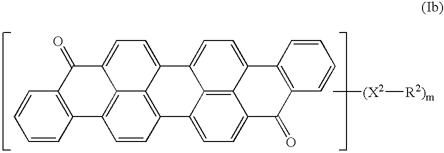

as markers for liquids, in particular mineral oils, where the variables are:
X¹, X² are each independently —O—, —S—, —NH—, —NY—, —CO—, —O—CO—, —CO—O—, —S—CO—, —CO—S—, —NH—CO—, —CO—NH—, —NY—CO—, —CO—NY—, —CH₂—NH—, —CH₂—NY—, —CH₂—NH—CO— or —CH₂—NY—CO—, where the latter four groups mentioned are each bonded via the CH₂ group to the basic dibenzanthrone or isodibenzanthrone structure,
R¹, R², Y are each independently
   C₁-C₂₀-alkyl which is optionally interrupted by from 1 to 4 oxygen atoms in ether function;
   C₅-C₇-cycloalkyl which is optionally substituted by one or more C₁-C₂₀-alkyl groups which are optionally interrupted by from 1 to 4 oxygen atoms in ether function; saturated heterocyclic five- or six-membered radical which is optionally substituted by one or more C₁-C₂₀-alkyl groups which are optionally interrupted by from 1 to 4 oxygen atoms in ether function;
   C₆-C₁₀-aryl which is optionally substituted by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino;
   heteroaryl which has from 3 to 12 carbon atoms and may optionally be substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino;
   C₆-C₁₀-aryl-C₁-C₄-alkyl which is optionally substituted in the aryl radical by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino;
   or
   heteroaryl-C₁-C₄-alkyl having from 3 to 12 carbon atoms in the heteroaryl radical, the latter optionally being substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino,
   and
n, m are integers from 1 to 16, where, when n>1 or m>1, the n (X′—R¹) moieties or the m (X²—R²) moieties may be the same or different.

The present invention further relates to fuel and lubricant additive concentrates which comprise at least one dibenzanthrone derivative of the general formula Ia or an isodibenzanthrone derivative of the general formula Ib, and also to mineral oils which comprise at least one dibenzanthrone derivative of the general formula Ia or an isodibenzanthrone derivative of the general formula Ib or inventive fuel and lubricant additive concentrates.

Mineral oil is usually additized using additive concentrates (also referred to herein below, following the relevant terminology, as packages) which, in addition to a carrier oil and a mixture of different fuel additives, generally also comprise dyes and also, for invisible fiscal or manufacturer-specific marking, additionally markers. These packages enable the supply of different mineral oil distributors from a pool of unadditized mineral oil, to which the company-specific additization, color and marker are imparted only, for example, while the mineral oil is being transferred into appropriate transport containers, with the aid of their individual packages.

The markers for liquids and especially for mineral oils are usually substances which are either extracted from the liquid or the mineral oil and subsequently converted to colored compounds by derivatization, or substances which exhibit absorption either in the visible or in the invisible wavelength region of the spectrum (for example in the NIR).

Markers which have been proposed and find use per se, i.e. not just after preceding derivatization, include a very wide range of compound classes, for example phthalocyanines, naphthalocyanines, nickel-dithiolene complexes, aminium compounds of aromatic amines, methine dyes and azulenesquaric acid dyes (for example WO 94/02570 A1, WO 96/10620 A1), but also bisazo dyes (for example EP 256 460 A1).

Anthraquinone derivatives for coloring gasoline or mineral oils are described in the documents U.S. Pat. No. 2,611,772, U.S. Pat. No. 2,068,372, EP 1 001 003 A1 and EP 1 323 811 A2.

Mono-alkyl-substituted isodibenzanthrone derivatives for marking of liquid hydrocarbons have been proposed in the document U.S. Pat. No. 4,278,444, although only one representative of this compound class is listed with the designation “Color No. 131 Super Concentrate”. However, in-house structural investigations which had already been carried out at an earlier date indicate that the product of the aforementioned designation is not an isodibenzanthrone derivative but rather a carbonyl-free isodibenzanthracene derivative.

Fluorescent isodibenzanthracene and dibenzanthracene derivatives (these compounds are also referred to hereinbelow as “(iso)dibenzanthracenes” and their corresponding diketo compounds as “(iso)dibenzanthrones”) and their preparation from suitably substituted (iso)dibenzanthrone precursor compounds are described in the document U.S. Pat. No. 6,215,008. Also indicated therein is the possible use of the (iso)dibenzanthracene derivatives for marking individual liquid streams in complex plants with a multitude of liquid-conducting pipelines, and also generally for marking liquids, especially mineral oils.

Many of the aforementioned markers are sufficiently stable in the pure liquids which they are intended to mark and in the normally low concentrations in which they are present. However, when further ingredients are present in addition to the markers, undesired degradation reactions of the marker may occur—the marking is lost.

This also applies to the markers typically present in mineral oil products, and to the packages to an even greater degree. The latter are additive concentrates which, in addition to a carrier oil and a mixture of different fuel additives, generally also comprise dyes and also, for invisible fiscal or manufacturer-specific marking, additionally markers. These packages enable the supply of different mineral oil distributors from a pool of unadditized mineral oil, to which the company-specific additization, color and marker are imparted only, for example, while the mineral oil is being transferred into appropriate transport containers, with the aid of their individual packages.

Many of the common markers, including the (iso)dibenzanthracene derivatives listed in the aforementioned document, are sufficiently stable under the dilute conditions in the mineral oil, but not under the concentrated conditions in the packages; the action of the package components can change the characteristics (e.g. extinction) of the markers in an undesired manner within a very short time.

It was thus an object of the present invention to provide markers which feature very good long-term stability in the liquids to be marked, especially mineral oils, in particular in the presence of further components present in the liquids, especially mineral oils.

Accordingly, the compounds of the formulae Ia and Ib listed at the outset have been found for use as markers for liquids, especially mineral oils.

When the linking X¹ and X² groups in the formulae Ia and Ib are defined as—O—CO—, —CO—O—, —S—CO—, —CO—S—, —NH—CO—, —CO—NH—, —NY—CO— or —CO—NY—, they may be bonded to the basic (iso)dibenzanthrone structure either via the carbonyl group or via the heteroatom. It is additionally possible that a portion of the n linking X¹ groups and m linking X² groups listed above in one and the same molecule is bonded via the carbonyl group, and another portion via the heteroatoms.

All n X¹ groups and m X² groups are preferably bonded to the basic (iso)dibenzanthrone structure either via the carbonyl group or via the heteroatom.

When the linking X¹ and X² groups in the formulae Ia and Ib are defined as —CH₂—NH—, —CH₂—NY—, —CH₂—NH—CO— or —CH₂—NY—CO—, they are bonded to the basic (iso)dibenzanthrone structure via the CH₂ group.

Generally, the n X¹ groups and m X² groups in the particular formulae Ia and Ib may be different from one another.

Preferably, the n X¹ groups and m X² groups in the particular formulae Ia and Ib are the same and, where different bondings to the basic (iso)dibenzanthrone structure are possible, bonded in the same way.

Generally, the n R¹ radicals and m R² radicals in the particular formulae Ia and Ib may be different from one another.

Preferably the n R¹ groups and m R² groups in the particular formulae Ia and Ib are the same.

Generally, the n X¹R¹ and m X²R² moieties in the particular formulae Ia and Ib may be different from one another. In particular, it is possible in this context for both the linking groups X¹ and X² in the particular formulae Ia and Ib to be different from one another with regard to the chemical nature and also, in the given case, in the way in which they are bonded to the basic (iso)dibenzanthrone structure, and for the R¹ and R² radicals to be different from one another with regard to their chemical nature.

Preferably, the n X¹R¹ and m X²R² moieties in the particular formulae Ia and Ib are the same. This means that the particular n identical R¹ radicals and m identical R² radicals are bonded in the same way via the linking X¹ and X² groups to the basic (iso)dibenzanthrone structure when there are different methods of bonding for the latter groups.

Possible definitions of the variables R¹, R² and Y are:

C₁-C₂₀-Alkyl which is optionally interrupted by from 1 to 4 oxygen atoms in ether function is, for example, methyl, ethyl, propyl, isopropyl, butyl, isobutyl, sec-butyl, tert-butyl, pentyl, isopentyl, neopentyl, tert-pentyl, hexyl, 2-methylpentyl, heptyl, hept-3-yl, octyl, 2-ethylhexyl, isooctyl, nonyl, isononyl, decyl, isodecyl, undecyl, dodecyl, tridecyl, 3,5,5,7-tetramethylnonyl, isotridecyl (the above terms isooctyl, isononyl, isodecyl and isotridecyl are trivial names and stem from the alcohols obtained by the oxo process—on this subject, cf. Ullmanns Encyklopädie der technischen Chemie, 4th Edition, Volume 7, pages 215 to 217, and also Volume 11, pages 435 and 436), tetradecyl, pentadecyl, hexadecyl, heptadecyl, octadecyl, nonadecyl, eicosyl, methoxymethyl, 2-ethylhexoxymethyl, 2-methoxyethyl, 2-ethoxyethyl, 2-propoxyethyl, 2-isopropoxyethyl, 2-butoxyethyl, 2- or 3-methoxypropyl, 2- or 3-ethoxypropyl, 2- or 3-propoxypropyl, 2- or 3-butoxypropyl, 2- or 4-methoxybutyl, 2- or 4-ethoxybutyl, 2- or 4-propoxybutyl, 2- or 4-butoxybutyl, 3,6-dioxaheptyl, 3,6-dioxaoctyl, 4,8-dioxanonyl, 3,7-dioxaoctyl, 3,7-dioxanonyl, 4,7-dioxaoctyl, 4,7-dioxanonyl, 4,8-dioxadecyl, 3,6,8-trioxadecyl, 3,6,9-trioxaundecyl, 3,6,9,1 2-tetraoxatridecyl or 3,6,9,1 2-tetraoxatetradecyl. C₅-C₇-Cycloalkyl radicals are cyclopentyl, cyclohexyl and cycloheptyl. These cycloalkyls are optionally substituted by one or more, in particular up to three, C₁-C₂₀-alkyl groups, and the latter may optionally be interrupted by from 1 to 4 oxygen atoms in ether function. Examples of such C₁-C₂₀-alkyl groups optionally interrupted by oxygen atoms have already been listed above.

Saturated, heterocyclic five- or six-membered radicals which are optionally substituted by one or more C₁-C₂₀-alkyl groups which are optionally interrupted by from 1 to 4 oxygen atoms in ether function are derived, for example, from pyrrolidine, 2- or 3-methylpyrrolidine, 2,4-dimethyl-3-ethylpyrrolidine, pyrazolidine, 2-, 3-, 4- or 5-methylpyrazolidine, imidazolidine, 2-, 3-, 4- or 5-methylimidazolidine, oxazolidine, 2-, 4- or 5-methyloxazolidine, isoxazolidine, 3-, 4- or 5-methylisoxazolidine, piperidine, 2-, 3-, 4-methyl- or -ethylpiperidine, 2,6-dimethylpiperidine, piperazine, 4-(C₁-C₄-alkyl)piperazine such as 4-methyl- or 4-ethylpiperazine, morpholine, thiomorpholine or thiomorpholine S,S-dioxide.

C₆-C₁₀-Aryls are in particular phenyl and naphthyl. These are optionally substituted by one or more halogen such as fluorine, chlorine or bromine, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino. Appropriate C₁-C₂₀-alkyl radicals which are optionally interrupted by from 1 to 4 oxygen atoms in ether function, and C₁-C₂₀-alkyl radicals which are present in the C₁-C₂₀-alkoxy, C₁-C₂₀-alkylamino or C₁-C₂₀-dialkylamino groups, have already been listed above by way of example.

Heteroaryl radicals which have from 3 to 12 carbon atoms and are optionally substituted by one or more C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino are derived, for example, from pyrrole, furan, thiophene, pyrazole, isoxazole, isothiazole, imidazole, 1H-1,2,3-triazole, 1H-1,2,4-triazole, pyridine, pyrazine, pyridazine, 1H-azepine, 2H-azepine, oxazole, thiazole, 1,2,3-, 1,2,4- or 1,3,4-oxadiazole, 1,2,3-, 1,2,4- or 1,3,4-thiadiazole and also optionally the benzo or dibenzofused rings, for example quinoline, isoquinoline, indole, benzo[b]furan (coumarone), benzo[b]thiophene (thionaphthene), carbazole, dibenzofuran, dibenzothiophene, 1H-indazole, indoxazole, benzo[d]isothiazole, anthranil, benzimidazole, benzoxazole, benzothiazole, cinnoline, phthalazine, quinazoline, quinoxaline or phenazine. Appropriate C₁-C₂₀-alkyl substituents which are optionally interrupted by from 1 to 4 oxygen atoms in ether function and C₁-C₂₀-alkyl radicals which are present in the C₁-C₂₀-alkoxy, C₁-C₂₀-alkylamino or C₁-C₂₀-dialkylamino substituents have already been listed above by way of example.

C₆-C₁₀-Aryl-C₁-C₄-alkyls which are optionally substituted in the aryl radical by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino are in particular benzyl, phenylethyl, 3-phenylpropyl and 4-phenylbutyl. Appropriate C₁-C₂₀-alkyl radicals which are optionally interrupted by from 1 to 4 oxygen atoms in ether function, and C₁-C₂₀-alkyl radicals which are present in the C₁-C₂₀-alkoxy, C₁-C₂₀-alkylamino or C₁-C₂₀-dialkylamino groups, have already been listed above by way of example.

Heteroaryl-C₁-C₄-alkyls having from 3 to 12 carbon atoms in the heteroaryl radical, the latter optionally being substituted by one or more C₁-C₂₀-alkyl which is optionally interrupted by from 1 to 4 oxygen atoms in ether function, C₁-C₂₀-alkoxy, C₁-C₂₀-alkylamino or C₁-C₂₀-dialkylamino are derived, for example, from the heteroaryl radicals specified above which are bonded to the C₁-C₄-alkyl radicals either via a carbon atom or a heteroatom of the heteroaryl which is suitable for bonding. Appropriate C₁-C₂₀-alkyl radicals which are optionally interrupted by from 1 to 4 oxygen atoms in ether function, and C₁-C₂₀-alkyl radicals which are present in the C₁-C₂₀-alkoxy, C₁-C₂₀-alkylamino or C₁-C₂₀-dialkylamino groups have already been listed above by way of example.

Preference is given to using those compounds in which the variables in the formulae Ia and Ib are
X¹, x² are each independently —O—, —S—, —NH—, —NY—, —CO—,—O—CO—, —CO—O—, —S—CO—, —CO—S—, —NH—CO—, —CO—NH—, —NY—CO— or —CO—NY—,
R¹, R², Y are each independently
   C₁-C₁₅-alkyl which is optionally interrupted by from 1 to 4 oxygen atoms in ether function;
   cyclohexyl which is optionally substituted by one or more C₁-C₁₅-alkyl groups which are optionally interrupted by from 1 to 4 oxygen atoms in ether function;
   saturated heterocyclic five- or six-membered radical which is optionally substituted by one or more C₁-C₁₅-alkyl groups which are optionally interrupted by from 1 to 4 oxygen atoms in ether function;
   C₆-C₁₀-aryl which is optionally substituted by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino;
   heteroaryl which has from 3 to 5 carbon atoms and is optionally substituted by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino;
   phenyl-C₁-C₄-alkyl which is optionally substituted in the phenyl radical by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino;
   or
   heteroaryl-C₁-C₄-alkyl having from 3 to 5 carbon atoms in the heteroaryl radical, the latter optionally being substituted by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino,
   and
   n, m are integers from 1 to 8, where, when n>1 or m>1, the n (X¹—R¹) moieties or the m (X²—R²) moieties may be the same or different.

Particular preference is given to using those compounds in which the variables in the formulae Ia and Ib are
X¹, X² are each independently —O—, —NH—, —NY—, —CO—,—O—CO—, —CO—O—, —NH—CO—, —CO—NH—, —NY—CO— or —CO—NY—,
R¹, R², Y are each independently
   C₁-C₁₅-alkyl;
   cyclohexyl which is optionally substituted by one or more C₁-C₁₅-alkyl groups;
   saturated heterocyclic five- or six-membered radical which is optionally substituted by one or more C₁-C₁₅-alkyl groups;
   phenyl which is optionally substituted by one or more C₁-C₁₅-alkyl, C₁-C₁₅-alkoxy, C₁-C₁₅-alkylamino or di(C₁-C₁₅)alkylamino;
   heteroaryl which has from 3 to 5 carbon atoms and is optionally substituted by one or more C₁-C₁₅-alkyl, C₁-C₁₅-alkoxy, C₁-C₁₅-alkylamino or di(C₁-C₁₅)alkylamino;
   phenyl-C₁-C₄-alkyl which is optionally substituted in the phenyl radical by one or more C₁-C₁₅-alkyl, C₁-C₁₅-alkoxy, C₁-C₁₅-alkylamino or di(C₁-C₁₅)alkylamino;
   or
   heteroaryl-C₁-C₄-alkyl having from 3 to 5 carbon atoms in the heteroaryl radical, the latter optionally being substituted by one or more C₁-C₁₅-alkyl, C₁-C₁₅-alkoxy, C₁-C₁₅-alkylamino or di(C₁-C₁₅)alkylamino, and
   n, m are integers from 1 to 8, where, when n>1 or m>1, the n (X¹—R¹) moieties or the m (X²—R²) moieties may be the same or different.

The compounds Ia and Ib which are used with preference and particular preference as R¹, R² and Y radicals have already also been listed above by way of example.

Advantageously used in accordance with the invention are those (iso)dibenzanthrones and their preferred embodiments in which n and m assumes values of from 1 to 4, in particular values of 2 or 3.

The compounds of the formulae Ia and Ib to be used in accordance with the invention can be prepared by customary methods of organic synthesis, and it is advisable to start from corresponding reactants having basic (iso)dibenzanthrone structure.

For example, the alkoxy-substituted (iso)dibenzanthrones can be prepared from the corresponding hydroxy compounds by etherification under the customary conditions known to the those skilled in the art. The preparation of 3,4,11,12-tetra-hydroxydibenzanthrone and 1,2,10,11-tetrahydroxyisodibenzanthrone as possible starting compounds are described, for example, in K. S. Nair, K. H. Shah, Bull. Chem. Soc. Japan, 39, (1966), 2023-2026. The preparation of further alkoxy-substituted and also substituted (iso)dibenzanthrones which comprise other linking X¹ and X² groups in the substituents is described, for example, in U.S. Pat. No. 4,486,587.

Alkylthio-, arylthio-, alkylamino-, dialkylamino- and arylamino-substituted (iso)dibenzanthrones are obtainable, for instance, by reacting the appropriate halogenated, typically chlorinated or brominated, or nitrated compounds with the particular alkylthiols, arylthiols, alkylamines, dialkylamines or arylamines under the customary conditions of nucleophilic aromatic substitution known to those skilled in the art. (Iso)dibenzanthrones which have —CH₂—NY—CO—R substituents are preparable by reacting the (iso)dibenzanthrones with paraformaldehyde and the corresponding amides (or lactams) in polyphosphoric acid or sulfuric acid. This can be effected, for example, in analogy to the methods which are described in the document EP 343 108 A2 and the prior German patent application 10 2004 003791.4 for the synthesis of correspondingly substituted phthalocyanines.

The —CH₂—NY—CO—R¹ and —CH₂—NY—CO—R² substituents can also be introduced by reacting the unsubstituted (iso)dibenzanthrones with the corresponding hydroxymethylated compounds in the presence of a concentrated acid (for example in analogy to the preparation of correspondingly substituted phthalocyanines as described in the prior aforementioned German patent application), and the hydroxymethylated compounds are obtainable by general methods known to those skilled in the art. The preparation of hydroxymethyllactams from the lactams is described, for example, in the documents U.S. Pat. No. 4,769,454 and U.S. Pat. No. 3,073,843. The reaction conditions (temperature, reaction time, concentration, excess of the hydroxymethyl compound, etc.) control the degree of substitution of the (iso)dibenzanthrones and thus their solubility. The degree of substitution can be determined, for example, by mass spectroscopy.

Liquids which can be marked in accordance with the invention with the (iso)dibenzanthrones of the formulae Ia and Ib and their preferred embodiments are in particular organic liquids, for example alcohols such as methanol, ethanol, propanol, isopropanol, butanol, isobutanol, sec-butanol, pentanol, isopentanol, neopentanol or hexanol, glycols such as 1,2-ethylene glycol, 1,2- or 1,3-propylene glycol, 1,2-, 2,3- or 1,4-butylene glycol, di- or triethylene glycol or di- or tripropylene glycol, ethers such as methyl tert-butyl ether, 1,2-ethylene glycol monomethyl or dimethyl ether, 1,2-ethylene glycol monomethyl or diethyl ether, 3-methoxypropanol, 3-isopropoxypropanol, tetrahydrofuran or dioxane, ketones such as acetone, methyl ethyl ketone or diacetone alcohol, esters such as methyl acetate, ethyl acetate, propyl acetate or butyl acetate, aliphatic or aromatic hydrocarbons such as pentane, hexane, heptane, octane, isooctane, petroleum ether, toluene, xylene, ethylbenzene, tetralin, decalin, dimethyinaphthalene, white spirit, mineral oil such as gasoline, kerosene, diesel oil or heating oil, natural oils such as olive oil, soya oil or sunflower oil, or natural or synthetic motor, hydraulic or gearbox oils, for example motor vehicle oil or sewing machine oil, or brake fluids.

In particular, the (iso)dibenzanthrones of the formulae Ia and Ib and their preferred embodiments are used to mark mineral oils.

It is of course also possible to add to the liquids, especially to the mineral oils, a plurality of different dibenzanthrones of the formula Ia and their preferred embodiments, a plurality of different isodibenzanthrones of the formula Ib and their preferred embodiments, or else mixtures of dibenzanthrones and isodibenzanthrones.

The (iso)dibenzanthrones of the formulae Ia and Ib and their preferred embodiments may also be used as a component in fuel and lubricant additive concentrates (“packages”) which, in addition to a carrier oil and a mixture of different fuel additives, generally also comprise dyes and additionally also markers.

Also claimed in the context of the present invention are therefore those fuel and lubricant additive concentrates which comprise at least one dibenzanthrone derivative of the general formula Ia or an isodibenzanthrone derivative of the general formula Ib or preferred embodiments of these (iso)dibenzanthrones listed above.

The components present in such packages are then in particular:
a) at least one dibenzanthrone of the formula Ia or an isodibenzanthrone of the formula Ib or preferred embodiments of these (iso)dibenzanthrones,
b) at least one carrier oil,
c) at least one additive selected from the group consisting of detergents,
dispersants and
valve seat wear-inhibiting additives,
d) and also, if appropriate, further additives and assistants.

The carrier oils used are typically viscous, high-boiling and in particular thermally stable liquids. They cover the hot metal surfaces, for example the intake valves, with a thin liquid film and thus prevent or delay the formation and deposition of decomposition products on the metal surfaces.

Carrier oils useful as component b) of the fuel and lubricant additive concentrates are, for example, mineral carrier oils (base oils), especially those of the Solvent Neutral (SN) 500 to 2000 viscosity class, synthetic carrier oils based on olefin polymers having M_{N}=from 400 to 1800, in particular based on polybutene or polyisobutene (hydrogenated or nonhydrogenated), on poly-alpha-olefins or poly(internal olefins) and also synthetic carrier oils based on alkoxylated long-chain alcohols or phenols. According to the invention, adducts, to be used as carrier oils, of ethylene oxide, propylene oxide and/or butylene oxide to polybutyl alcohols or polyisobutene alcohols are described, for instance, in EP 277 345 A1; further polyalkene alcohol polyalkoxylates to be used in accordance with the invention are described in WO 00/50543 A1. Further carrier oils to be used also include polyalkene alcohol polyether amines, as detailed in WO 00/61708.

It is of course also possible to use mixtures of different carrier oils, as long as they are compatible with one another and with the remaining components of the packages.

Carburetors and intake systems of internal combustion engines, but also injection systems for fuel metering, are being contaminated to an increasing degree by impurities which are caused, for example, by dust particles from the air and uncombusted hydrocarbons from the combustion chamber.

To reduce or prevent these contaminations, additives (“detergents”) are added to the fuel to keep valves and carburetors or injection systems clean. Such detergents are generally used in combination with one or more carrier oils. The carrier oils exert an additional “wash function”, support and often promote the detergents in their action of cleaning and keeping clean, and can thus contribute to the reduction in the amount of detergents required.

It should also be mentioned here that many of the substances typically used as carrier oils display additional action as detergents and/or dispersants, which is why the proportion of the latter can be reduced in such a case. Such carrier oils having detergent/dispersant action are detailed, for instance, in the last-mentioned WO document.

It is also often impossible to clearly delimit the mode of action of detergents, dispersants and valve seat wear-inhibiting additives, which is why these compounds are listed in summary under component c). Customary detergents which find use in the packages are listed, for example, in WO 00/50543 A1 and WO 00/61708 A1 and include:

polyisobuteneamines which are obtainable according to EP-A 244 616 by hydroformylation of highly reactive polyisobutene and subsequent reductive amination with ammonia, monoamines or polyamines, such as dimethyleneaminopropylamine, ethylenediamine, diethylenetriamine, triethylenetetramine or tetraethylenepentamine, poly(iso)buteneamines which are obtainable by chlorination of polybutenes or polyisobutenes having double bonds predominantly in the β- and γ-position and subsequent amination with ammonia, monoamines or the abovementioned polyamines,

poly(iso)buteneamines which are obtainable by oxidation of double bonds in poly(iso)butenes with air or ozone to give carbonyl or carboxyl compounds and subsequent amination under reducing (hydrogenating) conditions,

polyisobuteneamines which are obtainable according to DE-A 196 20 262 from polyisobutene epoxides by reaction with amines and subsequent dehydration and reduction of the amino alcohols,

polyisobuteneamines which optionally comprise hydroxyl groups and are obtainable according to WO-A 97/03946 by reaction of polyisobutenes having an average degree of polymerization P of from 5 to 100 with nitrogen oxides or mixtures of nitrogen oxides and oxygen and subsequent hydrogenation of these reaction products,

polyisobuteneamines which comprise hydroxyl groups and are obtainable according to EP-A 476 485 by reaction of polyisobutene epoxides with ammonia, monoamines or the abovementioned polyamines,

polyetheramines which are obtainable by reaction of C₂- to C₃₀-alkanols, C₆- to C₃₀-alkanediols, mono- or di-C₂- to C₃₀-alkylamines, C₁- to C₃₀-alkylcyclohexanols or C₁- to C₃₀-alkylphenols with from 1 to 30 mol of ethylene oxide and/or propylene oxide and/or butylene oxide per hydroxyl or amino group and subsequent reductive amination with ammonia, monoamines or the abovementioned polyamines, and also

“polyisobutene Mannich bases” which are obtainable according to EP-A 831 141 by reaction of polyisobutene-substituted phenols with aldehydes and monoamines or the abovementioned polyamines.

Further detergents and/or valve seat wear-inhibiting additives to be used are listed, for example, in WO 00/47698 A1 and include compounds which have at least one hydrophobic hydrocarbon radical having a number-average molecular weight (MN) of from 85 to 20 000 and at least one polar moiety, and which are selected from:
(i) mono- or polyamino groups having up to 6 nitrogen atoms, of which at least one nitrogen atom has basic properties;
(ii) nitro groups, optionally in combination with hydroxyl groups;
(iii) hydroxyl groups in combination with mono- or polyamino groups, in which at least one nitrogen atom has basic properties;
(iv) carboxyl groups or their alkali metal or alkaline earth metal salts;
(v) sulfonic acid groups or their alkali metal or alkaline earth metal salts;
(vi) polyoxy-C₂- to -C₄-alkylene groups which are terminated by hydroxyl groups, mono- or polyamino groups, in which at least one nitrogen atom has basic properties, or by carbamate groups;
(vii) carboxylic ester groups;
(viii) moieties derived from succinic anhydride and having hydroxyl and/or amino and/or amido and/or imido groups; and
(ix) moieties obtained by Mannich reaction of phenolic hydroxyl groups with aldehydes and mono- or polyamines.

Additives comprising mono- or polyamino groups (i) are preferably polyalkenemono- or polyalkenepolyamines based on polypropene or on highly reactive (i.e. having predominantly terminal double bonds, usually in the β- and γ-positions) or conventional (i.e. having predominantly internal double bonds) polybutene or polyisobutene having M_{N}=from 300 to 5000. Such additives based on highly reactive polyisobutene, which can be prepared from the polyisobutene (which may comprise up to 20% by weight of n-butene units) by hydroformylation and reductive amination with ammonia, monoamines or polyamines, such as dimethylaminopropylamine, ethylenediamine, diethylenetriamine, triethylenetetramine or tetraethylenepentamine, are disclosed in particular in EP 244 616 A2. When polybutene or polyisobutene having predominantly internal double bonds (usually in the β- and γ-positions) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be the same as those used above for the reductive amination of the hydroformylated highly reactive polyisobutene. Corresponding additives based on polypropene are described in particular in WO 94/24231 A1.

Further preferred additives comprising monoamino groups (i) are the hydrogenation products of the reaction products of polyisobutenes having an average degree of polymerization P of from 5 to 100 with nitrogen oxides or mixtures of nitrogen oxides and oxygen, as described in particular in WO 97/03946 A1.

Further preferred additives comprising monoamino groups (i) are the compounds obtainable from polyisobutene epoxides by reaction with amines and subsequent dehydration and reduction of the amino alcohols, as described in particular in DE 196 20 262 A1.

Additives comprising nitro groups (ii), optionally in combination with hydroxyl groups, are preferably reaction products of polyisobutenes having an average degree of polymerization P of from 5 to 100 or from 10 to 100 with nitrogen oxides or mixtures of nitrogen oxides and oxygen, as described in particular in WO 96/03367 A1 and WO 96/03479 A1. These reaction products are generally mixtures of pure nitropolyisobutanes (e.g. α,β-dinitropolyisobutane) and mixed hydroxynitropolyisobutanes (e.g. α-nitro-β-hydroxypolyisobutane).

Additives comprising hydroxyl groups in combination with mono- or polyamino groups (iii) are in particular reaction products of polyisobutene epoxides obtainable from polyisobutene having preferably predominantly terminal double bonds and M_{N}=from 300 to 5000, with ammonia or mono- or polyamines, as described in particular in EP 476 485 A1.

Additives comprising carboxyl groups or their alkali metal or alkaline earth metal salts (iv) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP 307 815 A1. Such additives serve mainly to prevent valve seat wear and can, as described in WO 87/01126 A1, advantageously be used in combination with customary detergents such as poly(iso)buteneamines or polyetheramines. Additives comprising sulfonic acid groups or their alkali metal or alkaline earth metal salts (v) are preferably alkali metal or alkaline earth metal salts of an alkyl sulfosuccinate, as described in particular in EP 639 632 A1. Such additives serve mainly to prevent valve seat wear and can be used advantageously in combination with customary detergents such as poly(iso)buteneamines or polyetheramines.

Additives comprising polyoxy-C₂-C₄-alkylene moieties (vi) are preferably polyethers or polyetheramines which are obtainable by reaction of C₂- to C₆₀-alkanols, C₆- to C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP 310 875 A1, EP 356 725 A1, EP 700 985 A1 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (vii) are preferably esters of mono-, di- or tricarboxylic acids with long-chain alkanols or polyols, in particular those having a minimum viscosity of 2 mm²/s at 100° C., as described in particular in DE 38 38 918 A1. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives which comprise moieties derived from succinic anhydride and have hydroxyl and/or amino and/or amido and/or imido groups (viii) are preferably corresponding derivatives of polyisobutenylsuccinic anhydride which are obtainable by reacting conventional or highly reactive polyisobutene having M_{N}=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. Such gasoline fuel additives are described in particular in U.S. Pat. No.4,849,572.

Additives comprising moieties obtained by Mannich reaction of phenolic hydroxyl groups with aldehydes and mono- or polyamines (ix) are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having M_{N}=from 300 to 5000. Such “polyisobutene-Mannich bases” are described in particular in EP 831 141 A1.

For a more precise definition of the additives detailed individually, reference is explicitly made here to the disclosures of the abovementioned prior art documents.

Dispersants as component c) are, for example, imides, amides, esters and ammonium and alkali metal salts of polyisobutenesuccinic anhydrides. These compounds find use especially in lubricant oils, but sometimes also as detergents in fuel compositions.

Further additives and assistants which may, if appropriate, be present as component d) of the packages are

organic solvents, for example alcohols such as methanol, ethanol, propanol, isopropanol, butanol, isobutanol, sec-butanol, pentanol, isopentanol, neopentanol or hexanol, for example glycols such as 1,2-ethylene glycol, 1,2- or 1,3-propylene glycol, 1,2-, 2,3- or 1,4-butylene glycol, di- or triethylene glycol or di- or tripropylene glycol, for example ethers such as methyl tert-butyl ether, 1,2-ethylene glycol monomethyl ether or 1,2-ethylene glycol dimethyl ether, 1,2-ethylene glycol monoethyl ether or 1,2-ethylene glycol diethyl ether, 3-methoxypropanol, 3-isopropoxypropanol, tetrahydrofuran or dioxane, for example ketones such as acetone, methyl ethyl ketone or diacetone alcohol, for example esters such as methyl acetate, ethyl acetate, propyl acetate or butyl acetate, for example lactams such as N-methylpyrrolidinone (NMP), for example aliphatic or aromatic hydrocarbons and also mixtures thereof such as pentane, hexane, heptane, octane, isooctane, petroleum ether, toluene, xylene, ethylbenzene, tetralin, decalin, dimethylnaphthalene or white spirit and, for example, mineral oil such as gasoline, kerosene, diesel oil or heating oil,

corrosion inhibitors, for example based on ammonium salts, having a tendency to form films, of organic carboxylic acids or of heterocyclic aromatics in the case of ferrous metal corrosion protection,

antioxidants or stabilizers, for example based on amines such as p-phenylene-diamine, dicyclohexylamine or derivatives thereof or on phenols such as 2,4-di-tert-butylphenol or 3,5-di-tert-butyl-4-hydroxyphenylpropionic acid,

demulsifiers,

antistats,

metallocenes such as ferrocene or methylcyclopentadienylmanganese tricarbonyl, lubricity improvers (lubricity additives) such as certain fatty acids, alkenylsuccinic esters, bis(hydroxyalkyl) fatty amines, hydroxyacetamides or castor oil,

amines for reducing the pH of the fuel,

further markers other than phthalocyanines of the formula I and their preferred embodiments and

dyes.

The concentration of component a), i.e. of the at least one dibenzanthrone of the formula Ia or isodibenzanthrone of the formula Ib or the preferred embodiment of this (iso)dibenzanthrone, in the packages is typically selected in such a magnitude that, after addition of the package to the mineral oil, the desired concentration of marker(s) is present therein. Typical concentrations of the markers in the mineral oil are, for instance, in the range from 0.01 up to a few 10s of ppm by weight.

Component b), i.e. the at least one carrier oil, is present in the packages typically in a concentration of from 1 to 50% by weight, in particular from 5 to 30% by weight, and component c), i.e. the at least one detergent and/or the at least one dispersant, typically in a concentration of from 25 to 90% by weight, in particular from 30 to 80% by weight, based in each case on the total amount of components a) to c) and, where present, d), the sum of the individual concentrations of components a) to c) and, if appropriate, d) adding up to 100% by weight.

When, as component d), corrosion inhibitors, antioxidants or stabilizers, demulsifiers, antistats, metallocenes, lubricity improvers and amines to reduce the pH of the fuel are present in the packages, the sum of their concentrations typically does not exceed 10% by weight, based on the total weight of the package (i.e. the total amount of components a) to c) and d)), the concentration of the corrosion inhibitors and demulsifiers being typically in the range of from in each case about 0.01 to 0.5% by weight of the total amount of the package.

When, as component d), additional organic solvents (i.e. not already introduced with the remaining components) are present in the packages, the sum of their concentrations typically does not exceed 20% by weight, based on the total amount of the package. These solvents generally stem from solutions of the markers and/or dyes, which are added to the packages instead of the pure markers and/or dyes with a view to more precise meterability.

When, as component d), further markers other than dibenzanthrones of the formula Ia, isodibenzanthrones of the formula Ib or their preferred embodiments are present in the packages, their concentration is in turn based on the content that they are to have after addition of the packages in mineral oil. That which was stated for component a) applies mutatis mutandis.

When, as component d), dyes are present in the inventive packages, their concentration is typically, for instance, between 0.1 to 5% by weight, based on the total amount of the package.

The present application further provides mineral oils which comprise at least one dibenzanthrone derivative of the general formula Ia or at least one isodibenzanthrone derivative of the general formula Ib or their preferred embodiments or else inventive fuel and lubricant additive concentrates comprising such (iso)dibenzanthrones of the formula Ia or Ib.

### EXAMPLES

Various (iso)dibenzanthrones were investigated with regard to their storage stability in the presence of fuel additives.

A) Preparation of the Compounds Used in Accordance with the Invention:

Compound 1: Extinction maximum in toluene: 620 nm

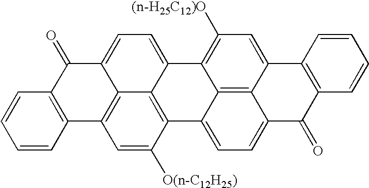

Compound 2: Extinction maximum in toluene: 620 nm

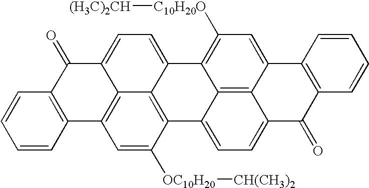

Compound 3: Extinction maximum in tetrahydrofuran: 642 nm

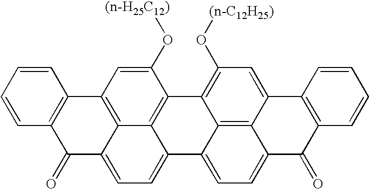

Synthesis of compound 1:

19.52 g (0.04 mol) of 6,15-dihydroxyisodibenzanthrone, 49.8 g (0.2 mol) of 1-dodecyl bromide (from Merck) and 11.04 g (0.08 mol) of potassium carbonate were introduced into 100 ml of N-methylpyrrolidone, and the reaction mixture was stirred at 130° C. for 6 hours. The mixture was allowed to cool to room temperature and diluted with 500 ml of ethanol, and the solid was filtered off with suction. 23.9 g (corresponding to a yield of 72% of theory) of product of the formula shown above were obtained.

Compounds 2 and 3 were prepared in an analogous manner using, respectively, 1-isotridecyl bromide and 6,15-dihydroxyisodibenzanthrone (compound 2) and 1-dodecyl bromide and 16,17-dihydroxydibenzanthrone.

Comparative compound (C): extinction maximum in Shellsol AB: 533 nm

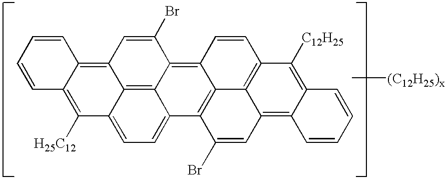

The comparative compound was prepared in accordance with Example 3 of the document U.S. Pat. No. 6,215,008.

B) Stability-Testing Storage in Mineral Oils at 40° C.:

50 mg of the particular compound were dissolved in 50 ml of Shellsol AB. Subsequently, the mixture was filtered through a fluted paper filter, 6.5 ml or 5.0 ml of the filtrate were introduced into 10 ml ampoules, the ampoule was made up to 10 ml (corresponding to a content of the particular compound of from 0.01 to 0.08%) with 3.5 ml of a commercial carrier oil or 5.0 ml of a detergent based on polyisobuteneamine (PIBA) (solution with PIBA content of 50% by weight), and the solutions were stored in the ampoules sealed air-tight at 40° C. in a water bath. After the storage times listed in the table which follows, samples were taken and analyzed in cuvettes having a diameter of 1 mm. In order to obtain better comparability of the storage experiments, the table lists extinctions normalized to the reference, i.e. the starting extinction of the unstored sample, as a function of the particular storage time.

| Experiment | | Storage time | Normalized | Extinction |
|---|---|---|---|---|
| (compound) | Additive | (h) | extinction | maximum (nm) |
| C a | Carrier oil | 0 | 1.00 | 534 |
| (C) | | 139 | 0.78 | |
| | | 624 | 0.57 | |
| 1a | Carrier oil | 0 | 1.00 | 620 |
| (1) | | 147 | 0.80 | |
| | | 795 | 0.86 | |
| 2a | Carrier oil | 0 | 1.00 | 618 |
| (2) | | 48 | 0.98 | |
| | | 499 | 0.91 | |
| 3a | Carrier oil | 0 | 1.00 | 637 |
| (3) | | 169 | 0.92 | |
| | | 341 | 0.89 | |
| | | 504 | 0.86 | |
| C b | PIBA | 0 | 1.00 | 533 |
| (C) | | 139 | 0.95 | |
| | | 624 | 0.92 | |
| 1b | PIBA | 0 | 1.00 | 616 |
| (1) | | 48 | 0.93 | |
| | | 336 | 0.93 | |
| | | 499 | 0.98 | |
| | | 686 | 1.00 | |
| 2b | PIBA | 0 | 1.00 | 584 |
| (2) | | 16 | 1.07 | |
| | | 136.5 | 1.05 | |
| | | 185 | 1.07 | |
| | | 473 | 1.08 | |
| 3b | PIBA | 0 | 1.00 | 634 |
| (3) | | 169 | 1.03 | |
| | | 341 | 1.01 | |
| | | 504 | 1.03 | |

It is evident from the above table that the color behavior, depending on the storage time, of the solutions of experiments 1 b to 3b does not differ substantially from that of the solution of experiment Cb, i.e., in the presence of PIBA, both the compounds 1 to 3 and the comparative compound have equally good storage stabilities; in the presence of carrier oil, in contrast, significantly better storage stabilities are found for the solutions of experiments 1a to 3a than in the case of the solution of experiment Ca.

N.B. The brief opening of the ampoules to take the samples led, owing to slight evaporation of the solvent, to slight concentration of the marker in the solution. As a result, somewhat too high an extinction value and thus also somewhat too high a normalized extinction value were determined throughout. This is noticeable in particular where no great color changes with storage time occur, and also explains the values greater than 1.00 in experiments 2b and 3b.

1. A marker for liquid comprising dibenzanthrone derivatives of the general formula Ia 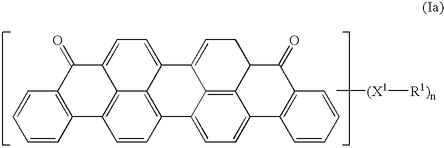
or isodibenzanthrone derivatives of the general formula Ib 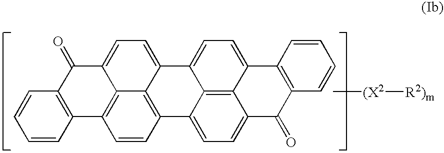
wherein the variables are:
X¹, X² are each independently —O—, —S—, —NH—, —NY—, —CO—,—O—CO—, —CO—O—, —S—CO—, —CO—S—, —NH—CO—, —CO—NH—, —NY—CO—, —CO—NY—, —CH₂—NH—, —CH₂—NY—, —CH₂—NH—CO— or —CH₂—NY—CO—, where the latter four groups mentioned are each bonded via the CH₂ group to the basic dibenzanthrone or isodibenzanthrone structure,
R¹, R², Y are each independently
C₁-C₂₀-alkyl which is optionally interrupted by from 1 to 4 oxygen atoms in ether function;
C₅-C₇-cycloalkyl which is optionally substituted by one or more C₁-C₂₀-alkyl groups which are optionally interrupted by from 1 to 4 oxygen atoms in ether function;
saturated heterocyclic five- or six-membered radical which is optionally substituted by one or more C₁-C₂0-alkyl groups which are optionally interrupted by from 1 to 4 oxygen atoms in ether function; C₆-C₁₀-aryl which is optionally substituted by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino; heteroaryl which has from 3 to 12 carbon atoms and may optionally be substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino;
C₆-C₁₀-aryl-C₁-C₄-alkyl which is optionally substituted in the aryl radical by one or more halogen, cyano, nitro, hydroxyl, amino, C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)-amino;
or
heteroaryl-C₁-C₄-alkyl having from 3 to 12 carbon atoms in the heteroaryl radical, the latter optionally being substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino,
and
n, m are integers from 1 to 16, where, when n>1 or m>1, the n (X¹—R¹) moieties or the m (X²—R²) moieties may be the same or different.

2. The marker according to claim 1, wherein the variables in the formulae Ia and Ib are
X¹, X² are each independently —O—, —S—, —NH—, —NY—, —CO—,—O—CO—, —CO—O—, —S—CO—, —CO—S—, —NH—CO—, —CONH—, —NY—CO— or —CO—NY—,
R¹, R², Y are each independently
C₁-C₁₅-alkyl which is optionally interrupted by from 1 to 4 oxygen atoms in ether function;
cyclohexyl which is optionally substituted by one or more C₁-C₁₅-alkyl groups which are optionally interrupted by from 1 to 4 oxygen atoms in ether function;
saturated heterocyclic five- or six-membered radical which is optionally substituted by one or more C₁-C₁₅-alkyl groups which are optionally interrupted by from 1 to 4 oxygen atoms in ether function;
C₆-C₁₀-aryl which is optionally substituted by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino;
heteroaryl which has from 3 to 5 carbon atoms and is optionally substituted by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino;
phenyl-C₁-C₄-alkyl which is optionally substituted in the phenyl radical by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino;
or
heteroaryl-C₁-C₄-alkyl having from 3 to 5 carbon atoms in the heteroaryl radical, the latter optionally being substituted by one or more C₁-C₁₅-alkyl which is optionally interrupted by from 1 to 4 oxygen atoms in ether function, C₁-C₁₅-alkoxy, C₁-C₁₅-alkylamino or di(C₁-C₁₅)alkylamino,
and
n, m are integers from 1 to 8, where, when n>1 or m>1, the n (X¹—R¹) moieties or the m (X²—R²) moieties may be the same or different.

3. The marker according to claim 1, wherein the variables in the formulae Ia and Ib are
X¹, X² are each independently —O—, —NH—, —NY—, —CO—,—O—CO—, —CO—O—, —NH—CO—, —CO—NH—, —NY—CO— or —CO—NY—,
R¹, R², Y are each independently
C₁-C₁₅-alkyl;
cyclohexyl which is optionally substituted by one or more C₁-C₁₅-alkyl groups;
saturated heterocyclic five- or six-membered radical which is optionally substituted by one or more C₁-C₁₅-alkyl groups;
phenyl which is optionally substituted by one or more C₁-C₁₅-alkyl, C₁-C₁₅-alkoxy, C₁-C₁₅-alkylamino or di(C₁-C₁₅)alkylamino;
heteroaryl which has from 3 to 5 carbon atoms and is optionally substituted by one or more C₁-C₁₅-alkyl, C₁-C₁₅-alkoxy, C₁-C₁₅-alkylamino or di(C₁-C₁₅)alkylamino;
phenyl-C₁-C₄-alkyl which is optionally substituted in the phenyl radical by one or more C₁-C₁₅-alkyl, C₁-C₁₅-alkoxy, C₁-C₁₅-alkylamino or di(C₁-C₁₅)alkylamino;
or
heteroaryl-C₁-C₄-alkyl having from 3 to 5 carbon atoms in the heteroaryl radical, the latter optionally being substituted by one or more C₁-C₁₅-alkyl, C₁-C₁₅-alkoxy, C₁-C₁₅-alkylamino or di(C₁-C₁₅)alkylamino,
and
n, m are integers from 1 to 8, where, when n>1 or m>1, the n (X¹—R¹) moieties or the m (X²—R²) moieties may be the same or different.

4. (canceled)

5. A fuel and lubricant additive concentrate comprising at least one dibenzanthrone derivative of the general formula Ia or an isodibenzanthrone derivative of the general formula Ib according to claim 1.

6. A mineral oil comprising at least one dibenzanthrone derivative of the general formula Ia or an isodibenzanthrone derivative of the general formula Ib according to claim 1.

7. A mineral oil comprising the concentrates according to claim 5.
